(19) United States
(12) Patent Application Publication
(10) Pub. No.: US 2019/0160231 A1
(43) Pub. Date: May 30, 2019

(54) DEVICES AND METHODS TO ASSIST IN LOCATING AN ARTERY AND GAINING PERCUTANEOUS ACCESS THERETO

(71) Applicants: Jae S. SON, Rolling Hills Estates, CA (US); PPS U.K. LIMITED, Glasgow (GB)

(72) Inventors: Gordon Dobie, Dumfries (GB); Jae S. Son, Rolling Hills Estates, CA (US)

(21) Appl. No.: 16/145,303

(22) PCT Filed: Apr. 1, 2016
(86) PCT No.: PCT/US16/25467

§ 371 Date: Sep. 28, 2018

(51) Int. Cl. A61M 5/42

(52) U.S. Cl. CPC A61M 5/427; A61M 2230/005; A61M 2205/583; A61M 2230/30

(57) ABSTRACT

A device (100, 200, 300) for identifying an arterial location includes a housing (110, 210, 310) with a tactile pressure sensor array (150, 250, 350) configured to detect pulsations of an artery once positioned over the presumed location of an artery. One of several LED lights (132, 232, 332) is energized to guide a clinician towards a preferred location of needle 10 insertion. The method of the invention comprises steps of positioning the device over the artery, reading pressure signals from individual pressure sensors, identifying peaks of pulsations and selecting the individual pressure sensor with the highest pulsation peaks as the closest to the artery located underneath. A corresponding LED light (132, 232, 332) is then activated to facilitate percutaneous access to the artery.

### CROSS-REFERENCE DATA

This US Patent Application claims a priority date benefit from a PCT Patent Application No. PCT/US16/25467 filed 1 Apr. 2016 with the same title, which is incorporated herein in its entirety by reference.

### BACKGROUND OF THE INVENTION

The present invention relates generally to devices to assist a clinician in locating and percutaneously puncturing an artery of a patient to gain arterial access. More particularly, the invention describes a device equipped with a tactile sensor array capable of detecting pulsations when positioned in a vicinity of an artery. The device further features an indicator of arterial location capable of pointing out a preferred location for a percutaneous puncture in order to gain access to the artery.

Arterial blood gas (ABG) sampling is a widely used procedure aimed at ascertaining a wide variety of questions about a patient. It typically includes withdrawing a small amount of blood for subsequent laboratory analysis. This blood sample provides valuable and time-critical information about a patient's gas exchange and acid base balance. Each year in Europe, 240 million arterial blood gas tests are performed. The arterial blood sample is typically taken from the radial artery in the patient's wrist by a trained clinician (typically an anesthetist, a junior doctor or a specialist nurse). The clinician typically uses their left hand to detect and feel for the pulsating artery. They then insert a suitably sized needle into the artery and extract a sample using their right hand. Importantly, a needle passes very closely to the clinician's left hand therefore increasing a risk of accidental puncture.

The existing arterial blood gas procedure has two issues: (1) Arteries are innervated, making the procedure painful. Patients sometimes react when the needle is inserted leading to the needle jumping out from the patient and potentially into the clinician causing a needle stick injury. Needle stick injuries are regrettably common, and can constitute a transmission path for blood borne infections such as Hepatitis and HIV. A large proportion of the patient group requiring an ABG are often critically unwell and agitated and/or confused as a result of their medical condition which in turn makes sampling more challenging and increases the risk of a needle stick injury. (2) The procedure can be challenging to clinicians, sometimes requiring them to insert the needle five or six times in order to be successful. Failed attempts increase patient discomfort and recovery time, increase infection risk, increase clinician anxiety, and erode patient confidence in their clinician.

There are other locations throughout the body that feature an arterial blood vessel located reasonably close to the skin and therefore suitable for a percutaneous puncture. Examples of such locations include brachial and axillary portions of the arterial vessel tree as well as a femoral artery. The present invention may be useful in these and possibly other locations to facilitate arterial access. In particular, rapid and accurate access to a femoral artery using a percutaneous needle may be difficult using a traditional manual palpation technique. Patients with low blood pressure, obesity or diseased arteries present a particularly difficult challenge—especially in potentially life-threatening emergencies (Myocardial Infarction, Diabetic Ketoacidosis). Delay in gaining femoral access or multiple attempts to obtain a blood gas sample from the femoral artery are documented to potentially increase post-procedural vascular complication. In the worst case, they impact morbidity and even mortality for critically-ill patients.

The need exists therefore for a device and a method allowing a simplified, more reliable sampling technique of the arterial blood minimizing the number of stick attempts, reduction in the time taken to obtain such access, increase the safety profile of this procedure and reduce patient discomfort.

### SUMMARY OF THE INVENTION

Accordingly, it is an object of the present invention to overcome these and other drawbacks of the prior art by providing novel devices and methods for assisting a clinician in finding an artery and facilitating a percutaneous access thereto.

It is another object of the present invention to provide novel devices and methods to improve clinician safety while performing a percutaneous needle puncture to gain access to an artery and reduce the risk of inadvertent needle stick.

It is a further object of the present invention to provide novel devices and methods to assist a clinician in gaining a percutaneous access to an artery even in cases when the clinician is not experienced in such procedures.

It is yet a further object of the present invention to provide novel devices and methods allowing a clinician to gain percutaneous access from the very first attempt whereby reducing trauma to the artery and subcutaneous tissues arising from multiple attempts at such percutaneous puncture.

The device of the invention includes a housing containing a tactile pressure sensor array with a plurality of individual pressure sensors. These sensors may be configured to provide pressure signal data in response to a tactile pressure imparted thereon. Once positioned over the artery or in a vicinity of a presumed arterial location, pressure signals from these individual pressure sensors are obtained by a control unit of the device, processed and analyzed to identify at least one or a few individual pressure sensors with the strongest peak of pulse waveforms, indicating their position to be closest to an underlying artery. A visual indicator such as a string of LED lights may be used to identify for a clinician a preferred location of a needle puncture—by energizing the light corresponding to a location of maximum pulsatility among the individual pressure sensors. The device may further include a needle guide to facilitate insertion of the needle into the tissue at this preferred location and at a preferred angle of insertion—so as to maximize the probability of gaining arterial access on the first attempt.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a general illustration of the first embodiment of the present invention in use.

FIG. 2 is another view of the same with the insert showing details of the design of the first embodiment of the present invention.

FIGS. 3a through 3e are various views of the first embodiment of the present invention from different angles.

FIG. 4 is a general view of the second embodiment of the present invention.

FIGS. 5a through 5c are various side and front views of the same.

FIG. 6 is a general view of the second embodiment of the invention while in use.

FIG. 7 is another view of the second embodiment in use with the insert showing details of the design and interaction with a percutaneous needle.

FIG. 8a is a view of the second embodiment from the back with the insert showing details of the tactile pressure sensor array.

FIGS. 8b through 8e show other views of the tactile pressure array and various embodiments thereof.

FIG. 9 shows a general view of the third embodiment of the present invention.

FIGS. 10a through 10d show views of the third embodiment from different angles and details of the design.

FIG. 11 shows the third embodiment of the invention while in use—as it is being placed on a wrist of a patient.

FIG. 12 shows the third embodiment of the invention after it has been placed on a wrist of a patient.

FIG. 13 shows the third embodiment while in use during the time of identifying the location of an artery and inserting a needle to gain percutaneous access thereto.

FIG. 14 shows the same from a different angle with the insert showing the details of the design of the device and its interaction with a percutaneous needle.

FIG. 15 shows a functional block-diagram of the device and its control unit.

FIG. 16 shows a block-diagram of signal processing steps undertaken by the device.

### DETAILED DESCRIPTION OF THE PREFERRED EMBODIMENT OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

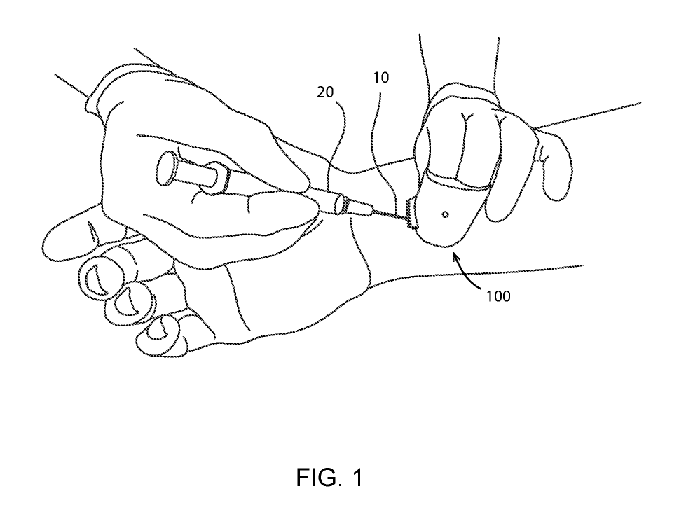

The device of the invention generally features a tactile pressure sensor array retained in a housing such that it can be pressed against the skin of the patient in a vicinity of a presumed arterial location. FIG. 1 shows a general overview of the first embodiment of the device 100 being positioned on a wrist of the patient. The device 100 may be retained against the skin with one or two fingers of the left hand by the clinician while the right hand may be used to hold a syringe 20 with the attached percutaneous needle 10.

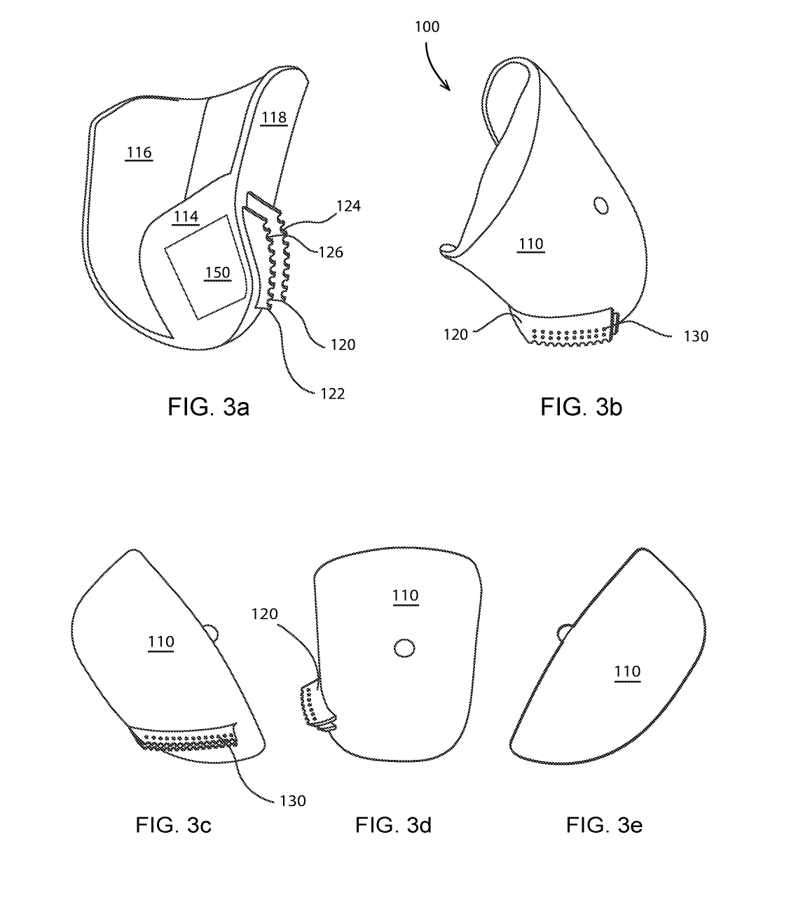

Details of the first embodiment of the present invention are depicted in FIGS. 1-3. The housing 110 is best seen in FIGS. 3a through 3e. It may be shaped to at least partially or completely go around two fingers of a human hand. In alternative embodiments, the housing 110 may also be shaped to go at least partially around one finger of a human hand, such as an index finger. The housing 110 may also be shaped as a shield to protect the fingers positioned inside thereof from an inadvertent needle puncture, which may be helpful in reducing the risk of transmitting an infection from the patient to the clinician.

To accomplish this, the housing 110 may feature two side walls 116 and 118 which together with the rest of the housing 110 may be shaped to wrap around the fingers of the clinician while in use. The housing 110 may also include a bottom portion 114 housing the tactile pressure sensor array 150 such that the sensitive surface of the sensor array is exposed to be in contact with the skin of the patient when the device is placed at a presumed location of an artery. The bottom portion 114 may be joined with the side wall 118 at the corner 112. Details of construction of the tactile pressure sensor array 150 are described in further parts of this specification.

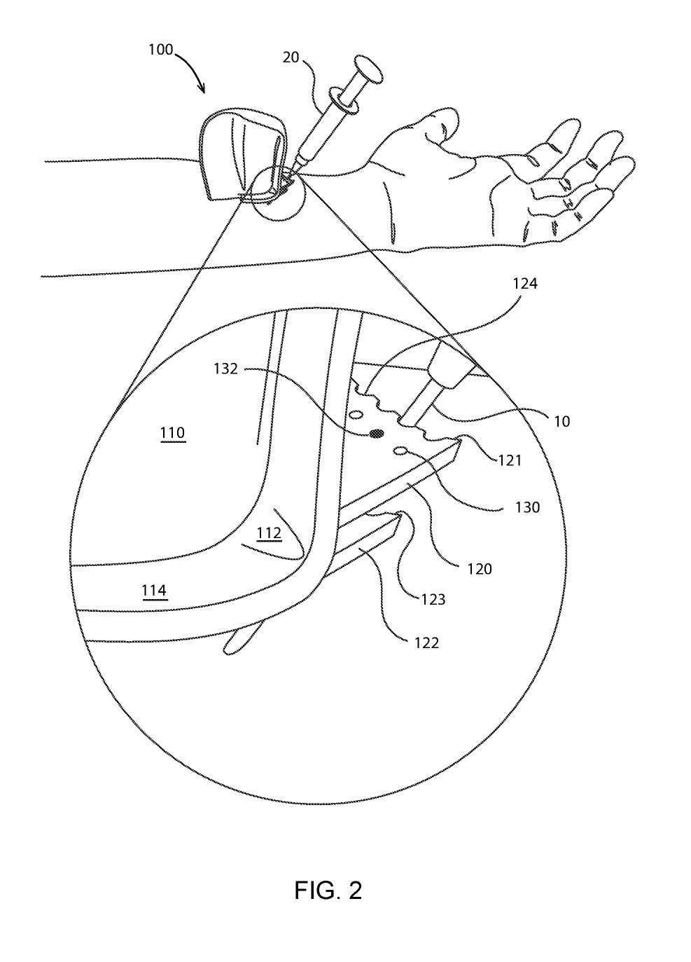

The device 100 may further feature a needle guide 120 with a first edge 121 configured to serve as a rest surface for the needle 10 during the needle puncture portion of the procedure. In embodiments, the first edge 121 may be straight (not shown) or contain a plurality of first notches 124, in which case each first notch 124 may be sized and shaped to retain the needle 10 therein during the process of advancing the needle towards the artery. In embodiments, the shape of each first notch 124 may be semi-circular as shown in FIG. 2. In other embodiments, such first notch 124 may be shaped as a triangle or even a full circle—in which case the needle may have to be passed therethrough—as the invention is not limited in this regard.

As can be appreciated by those skilled in the art, the location of the tactile pressure sensor array 150 is somewhat different from that of the first edge 121. Given a presumed and known thickness of the subcutaneous tissue layer on top of the artery, the position of the first edge 121 may be selected to be extended from the housing 110 and away from the tactile pressure sensor array 150 such that advancing the needle 10 towards the artery may proceed at a clinically acceptable angle of insertion.

To remove uncertainty as to the angle of needle 10 insertion, the device 100 may further include a second edge 123 as part of the second portion 122 of the needle guide 120. The distance of the second edge 123 from the housing 110 may be selected to be smaller than the distance of the first edge 121 from the same housing. In this case, the needle 10 would have a well-defined angle of insertion while resting on both the first edge and the second edge during the procedure.

A plurality of second notches 126 may be provided along the second edge 123. In embodiments, the second notches may be shaped and sized to be the same as the first notches. Furthermore, for each first notch 124 along the first edge 121 there may be provided a corresponding second notch 126 along the second notch 123.

As can be seen from the drawings, both the first edge 121 and an optional second edge 123 are positioned to be in parallel with at least one line of the pressure sensors of the tactile pressure sensor array 150. The orientation of one or both the first edge 121 and the second edge 123 may be generally across the projected path of the artery when the device 100 is in use. This is done so as to cause at least one or a few pressure sensors of the tactile pressure sensor array 150 to provide the highest peak amplitude pressure signal as compared with other surrounding pressure sensors. Once the pressure sensor with the peak signal is found, it is presumed to correspond in its location with the pulsating artery located underneath. Knowing the mechanical design of the housing and locations of the tactile pressure sensor array 150 and the first and second edges 121 and 123, it now becomes possible to indicate to the clinician the best place for a needle 10 along the first edge 121. It may also become possible to identify the preferred first notch 124 on which to rest the needle 10 in order to gain accurate access to the underlying artery.

The length of the first edge 121 and also the second edge 123 may be selected to traverse laterally a suitable area for needle puncture. In embodiments, such length may be selected to be from about ¼ inch to about 2 inches.

To further facilitate such indication of the preferred positioning of the needle, an indicator of arterial location 130 may be provided. In embodiments, such indicator of arterial location may be a plurality of light emitting diodes arranged in a single line, for example along the first edge 121. Other lights may also be used. In embodiments, LEDs 130 may be positioned each next to a corresponding first notch 124. In this case, a control unit of the device 100 may be configured to energize such light 132 at the preferred location of needle insertion so as to guide the clinician to use a particular notch 124 located next to the light 132 once it is turned on. The length of the indicator of arterial location 130 may be selected to be the same or similar to the length of the first edge 121. In embodiments such length may be from about ¼ inch to about 2 inches.

Alternative configurations of the indicator of arterial location 130 are also included in the scope of the present invention. Such alternatives may include a continuous line of lights, double line of LEDs as seen in FIG. 3b, a single point light source that can be adjusted (such as a laser beam which can be pointed at the preferred position for needle insertion), as well as an audible indicator.

In case of an audible indicator of arterial location, one alternative design for the device of the present invention may include a single notch on a needle guide and an audio emitter. In this case, the clinician may be instructed to position and re-position the device until the loudest sound is produced by the audible indicator—signifying that the proper place for the single notch of the needle guide with regard to the location of the artery underneath thereof has been achieved.

The use of the device is best depicted in FIGS. 1 and 2. According to the method of the present invention, obtaining a percutaneous access to an artery may include providing a device 100 for identifying arterial location. This device 100 as described above may comprise a tactile pressure sensor array 150 and an indicator of arterial location 130, which in turn may comprise a plurality of individual pressure sensors configured to detect a pressure signal corresponding to a tactile pressure imparted thereon.

The method of the invention may then include positioning of the device 100 near a presumed arterial location, such as over a wrist of the patient or over a groin area located above a projected path of a femoral artery. Once positioned on the skin of the patient, the device 100 may be slightly pushed towards the artery to assure intimate contact with the skin of the patient.

The method of the invention may then include a step of obtaining pressure signals from the plurality of individual pressure sensors of the tactile pressure sensor array 150. As the artery of the patient produces pulsations that may even be palpable by a human hand, tactile pressure would be applied to the sensitive surface of the individual pressure sensors of the array 150. Pressure signals produced by these individual pressure sensors may then be recorded and transmitted to the control unit of the device 100 for further processing, as described later in this specification.

According to the method of the invention, the control unit may be used to identify at least one or perhaps a few individual pressure sensors exhibiting the pressure signal having periodic peaks at a maximum level. Such individual pressure sensors are believed to be located in the closest proximity to the artery.

The device of the invention may further include a motion sensor to detect whether the device is in motion or stationary. Such sensor may be made as an accelerometer, a gyroscope or a combination thereof. The output signal of the motion sensor may be used by the control unit to determine the state of the device (stationary or moving) and use this determination in separating true pressure signal waveforms from motion artifacts, whereby further improving the accuracy of the arterial location determination.

Based on location of at least one individual pressure sensor identified in the previous step, the control unit may be configured to cause the indicator of arterial location 130 to identify a preferred location for a percutaneous needle puncture by energizing an LED 132 located next to the individual pressure sensors with the maximum peak of pulsations identified previously. The clinician may now proceed with inserting the needle 10 using the needle guide 120 and the energized LED 132.

In embodiments, the housing 110 may include provisions to accept a removable disposable cover or sheathing (not shown) so as to provide for sterile environment during use and satisfy infection control objectives. In other embodiments, the housing 110 may itself include snap-on portions serving as disposable covers. In further embodiments, the housing 110 may be configured to allow removable attachment of at least one or all of the tactile pressure sensor array 150, the needle guide 120, the indicator or arterial location 130 and the control unit with the microprocessor and other electronics. In further embodiments, the entire device 100 may be made to be disposable.

The shape of the housing shown in FIGS. 1-3 is designed for use with a left hand. It is contemplated that a symmetrically opposite shape and arrangement of components may be put together for using the device over the fingers of the right hand of the clinician.

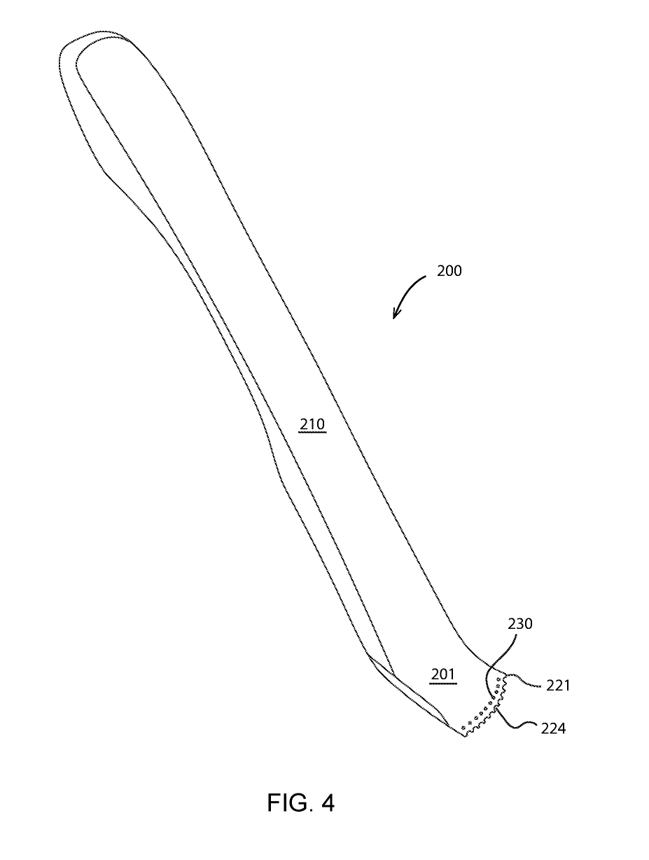

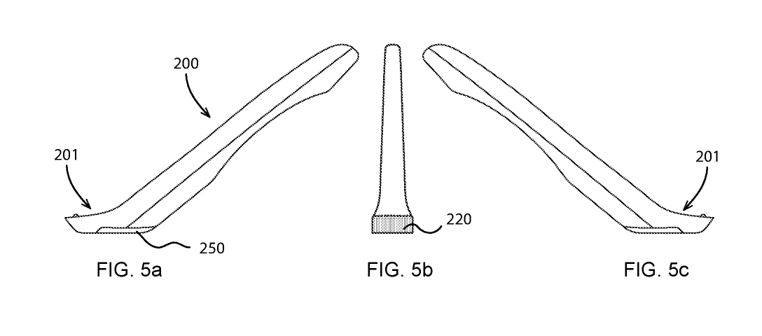

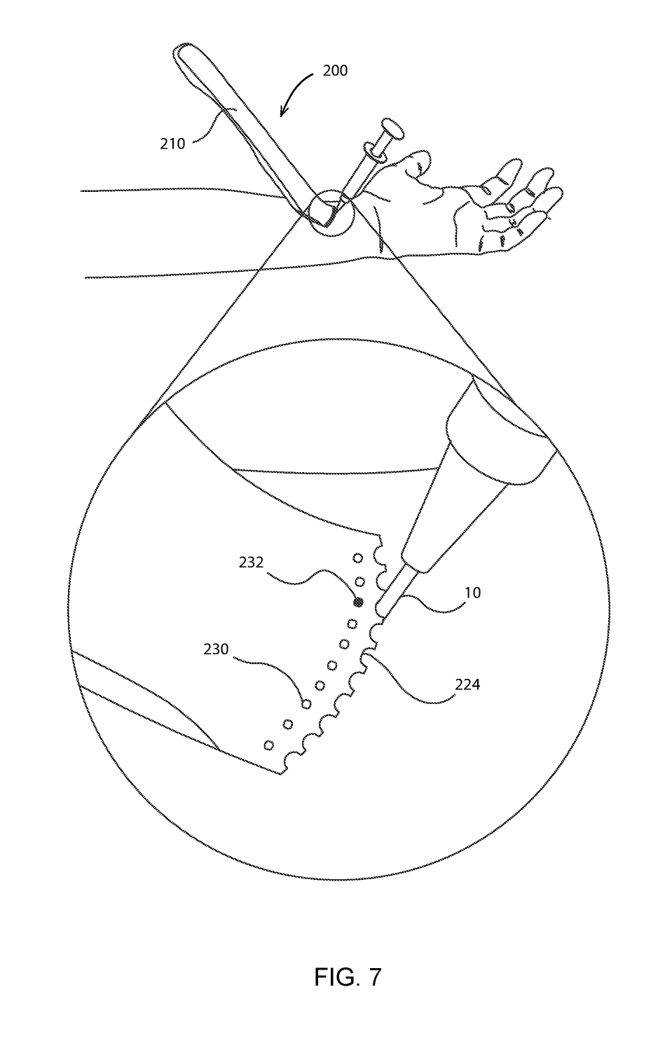

FIGS. 4 through 8 illustrate the second embodiment of the present invention. In this case, the device 200 has a similar working end (foot 201) as described above but may be retained against the skin of the patient using an elongated handle 210 shaped to be held by a human hand. The foot portion 201 of the device 200 may include a line of LED indicators 230 along a needle guide 220 featuring a first edge 221 having individual first notches 224 thereon. The handle 210 may contain the control unit of the device 200. Alternatively, the control unit may be housed in a separate housing and be connected to the handle 210 by a wire or even wirelessly. The foot 201 may also feature a tactile pressure sensor array 250 described in greater detail below.

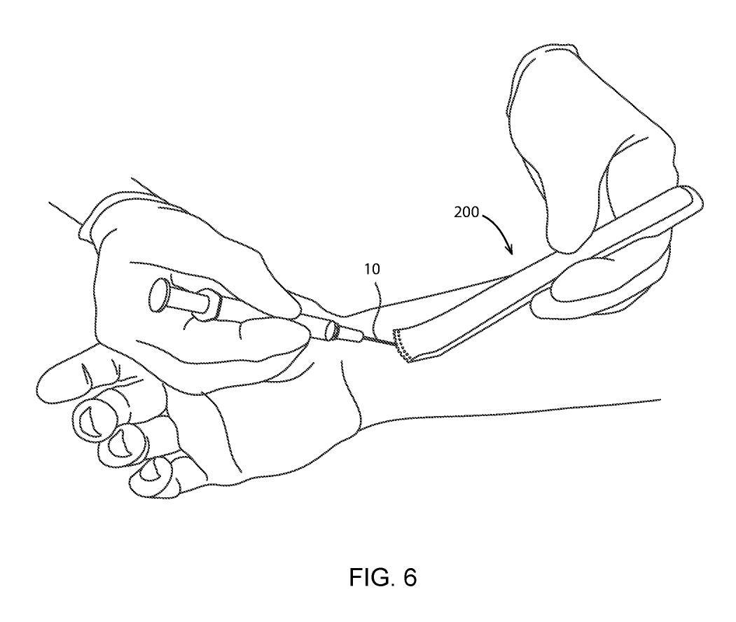

The use of the device 200 is seen best in FIG. 6 and includes positioning and retaining the device 200 such that the foot 201 is placed against the skin of the patient at a presumed location of the artery. Once the tactile pressure sensor array 250 is pressed against the skin, the individual pressure sensors with the highest peak pulsation signal is identified and the corresponding LED is energized so as to guide the clinician to use a particular notch 224 located over a preferred position for percutaneous puncture.

This configuration of the device has at least two advantages over the first embodiment, namely that the hand of the clinician is further removed from the needle 10 and that one device will be suitable for right-handed and left-handed clinicians. On the other hand, the first embodiment of the device allows at least some fingers of the clinician to remain over the artery and to confirm palpable pulsations at all times, which may be advantageous in case of loose skin or other uncertainties in certain clinical situations.

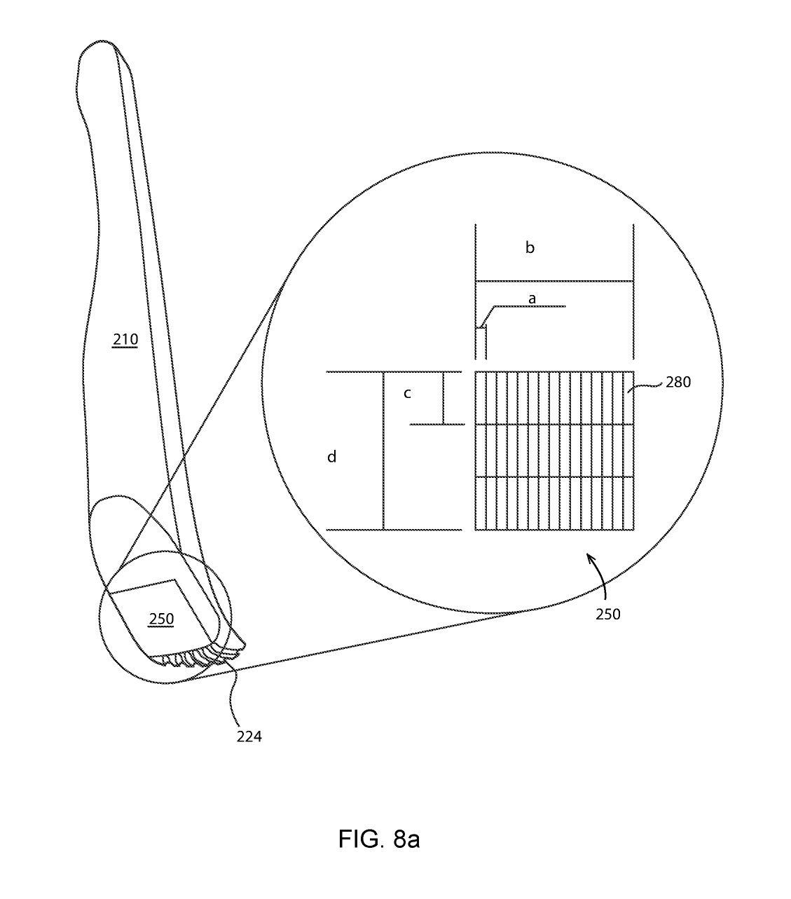

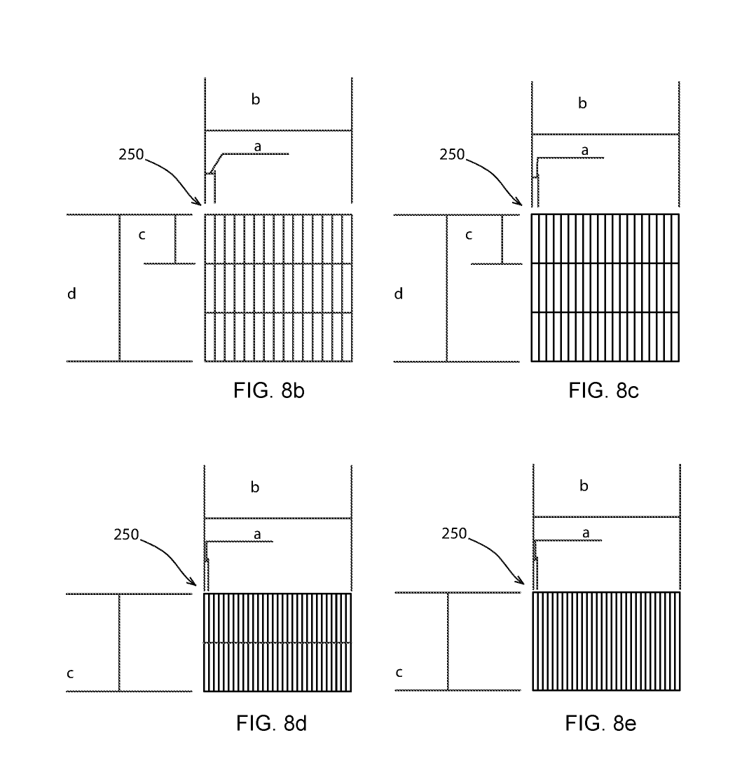

The tactile pressure sensor array 250 is now discussed in greater detail. This discussion pertains to all embodiments of the present invention. FIG. 8a shows a general outline of the tactile pressure sensor array 250. It may comprise one or more lines of individual pressure sensors 280. Each pressure sensor 280 may have a width “a”. The number of individual pressure sensors 280 in a single line may be “b”. Each line of individual pressure sensors may have a width “c” and the total width of the array may be “d”.

Individual pressure sensors may be as little as 0.02 inch by 0.02 inch. In embodiments, other individual pressure sensors may not be square and have dimensions of about 0.03 inch by about 0.20 inch. In yet other embodiments, each size of an individual pressure sensor can be as large as about 0.75 inch as the invention is not limited in this regard.

In embodiments, a single line of individual pressure sensors 280 may be used—as seen in FIG. 8e for example. At least three individual sensors may be included in such line. The number of individual pressure sensors 280 may be greater than 3 and include 5, 8, 10, 15, 20, 25, 30, 40, 50, 60, 70, 80, 90, 100 sensors or any other suitable number inbetween.

More than a single line of individual pressure sensors 280 may also be deployed. In embodiments, 2, 3, 4, 5, 6, 7, 8, 9, 10, 15, 20, 25, 30, 35, 40, or 50 lines of sensors may be deployed, some of these configurations are shown in FIGS. 8a through 8d. Individual pressure sensors 280 may be positioned to be adjacent to each other or may be spaced apart. In embodiments, adjacent individual pressure sensors may be spaced apart from each other from about 0.01 inches to about 0.40 inches inbetween.

Additional lines of sensors may be used to inform the clinician to move the device up or down the arm along the arterial path if the peak pressure is detected to be further away from the projected path of the needle guide. This may be done by using a blinking light rather than energizing a solid light—or with other suitable ways to communicate with the clinician.

Obviously, increased number of individual sensors 280 and higher density thereof may be beneficial to improve the resolution of the entire tactile pressure sensor array 250. However, greater number of sensors increase signal processing complexity and slows the sample rate.

Various types of individual pressure sensors and tactile pressure sensor arrays may be used for the purposes of the present invention. In embodiments, capacitive pressure sensor array may be formed using two electrode layers separated by a compressible dielectric layer. At least one or both electrode layers may be formed with parallel lines of conductive strips so as to form an overlapping matrix of electrodes. Imparting tactile pressure on one side of such array may cause local disturbance in capacitance which can be detected by the control unit. Details of such design may be found in numerous patents and patent applications by the inventors of the present invention, such as for example in U.S. Pat. Nos. 5,983,727; 7,378,856; 7,430,925; 7,609,178; 7,595,788; 8,400,402; 8,169,332; 8,127,623; 8,627,716—all such documents are incorporated herewith by reference in their respective entireties.

Other types of pressure sensors may also be used. In embodiments, piezo pressure sensors, piezo-resistive pressure sensors, optical pressure sensors, fiber-optic pressure sensors, resistive pressure sensors, electrical impedance pressure sensors, and quantum tunneling pressure sensors may be deployed as the invention is not limited in this regard.

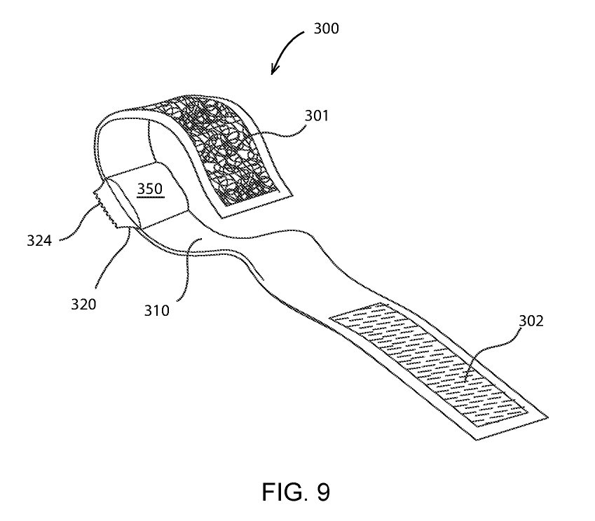

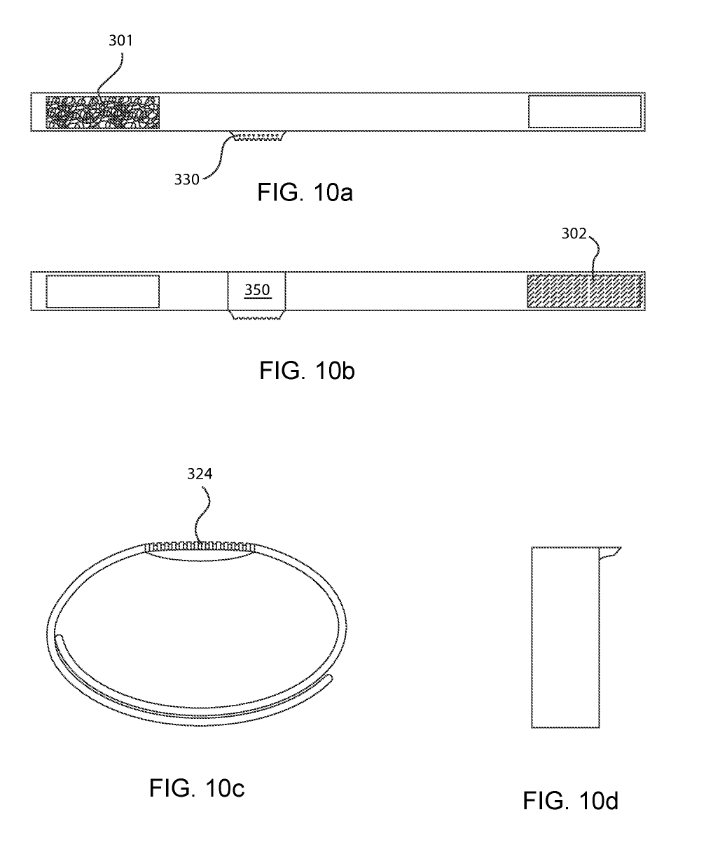

FIGS. 9 through 14 illustrate the third embodiment of the present invention configured to be placed about and secured at the wrist of the patient. The device 300 may be generally made as a watch. The housing 310 may have a pair of straps 301 and 302 that can be secured to each other with adjustable strap length—for example using a Velcro® hook-and-loop arrangement. The inside portion of the housing 310 may comprise a tactile pressure sensor array 350 positioned to face the skin of the patient when in use. The device 300 may further feature a needle guide 320 with the line of first notches 324 and the indicator of the arterial location 330, such as a line of LED lights along the notches 324.

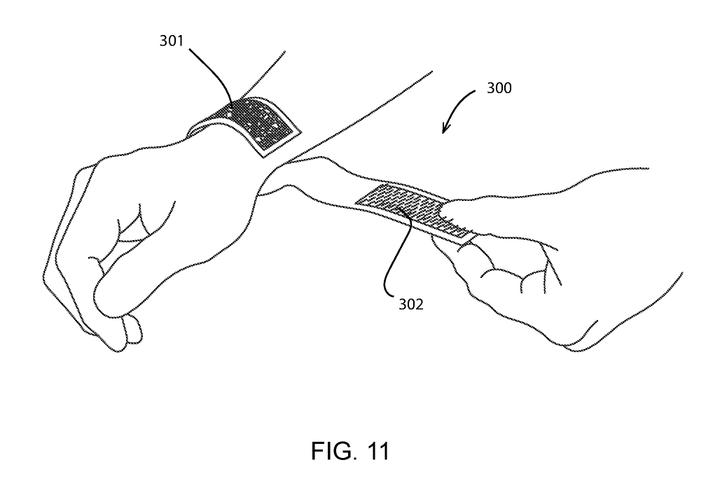

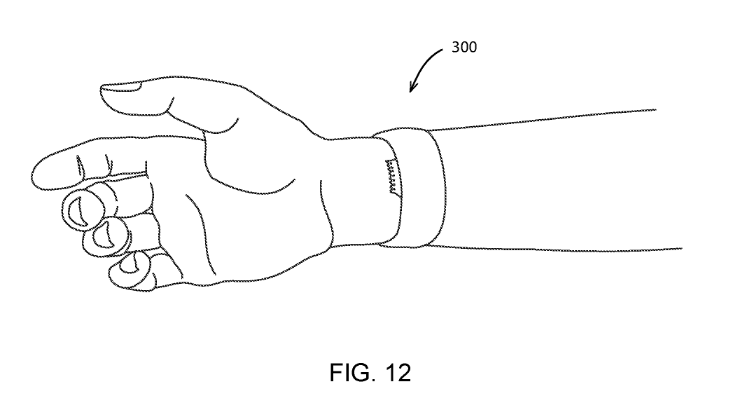

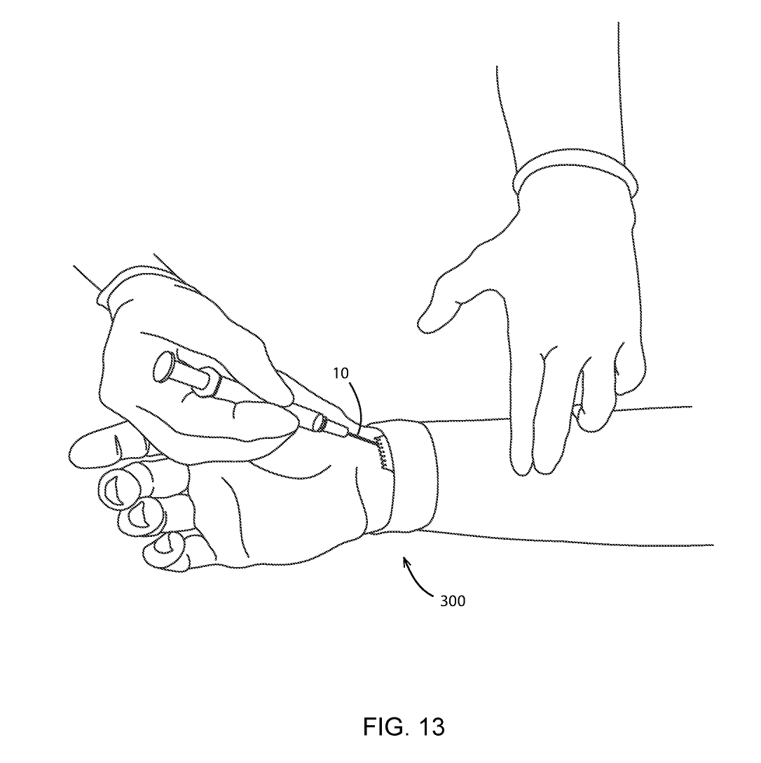

FIG. 11 shows positioning the device 300 on the patient. The straps 301 and 302 may be used to secure the device 300 in a watch-like manner. Care should be taken to assure the position of the tactile pressure sensor 350 facing the skin of the inner wrist of the patient—at a presumed location of the artery, as seen in FIG. 12. Securing the device 300 to the patient allows the clinician to proceed with finding the artery location and inserting the needle as seen in FIG. 13—with the other hand free to hold down the patients' arm.

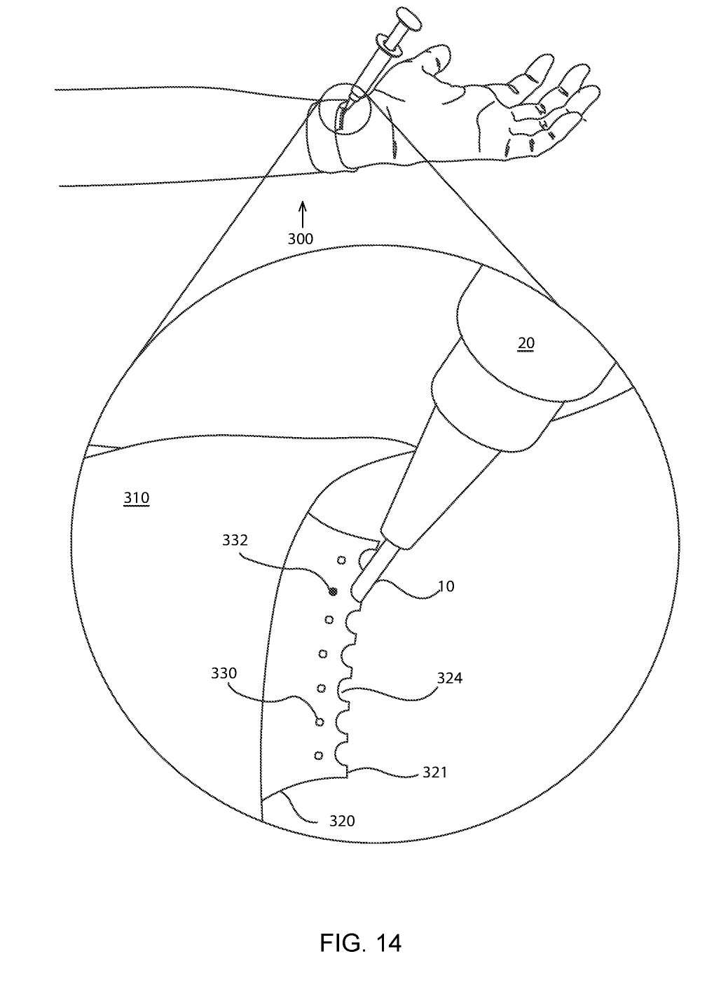

The third embodiment of the present invention features a similar needle guide 320 having a first edge 321 and a plurality of first edges 324 positioned across the LED lights 330 of the indicator of arterial position. Once the location of the artery has been found, the controller unit of the device may be configured to activate a closest light 332 to guide the clinician to insert the needle 10 using the notch located closest to the light 332—see FIG. 14.

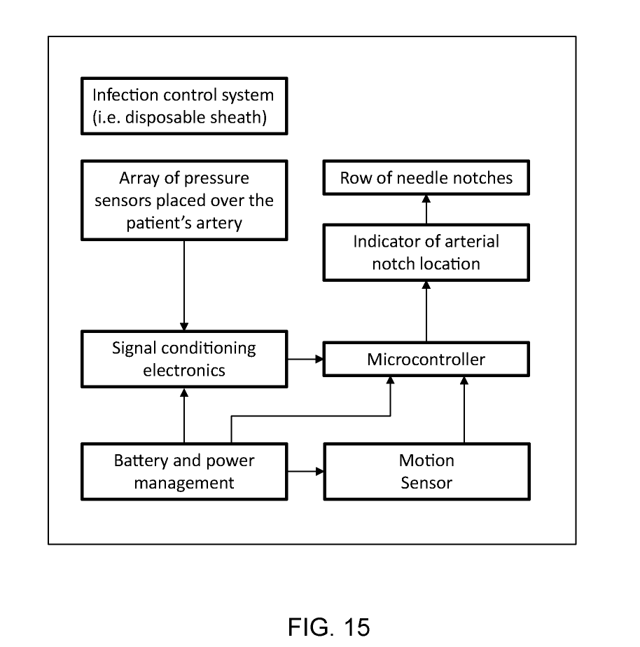

FIG. 15 shows a block-diagram of the major elements of the device of the present invention. As discussed earlier, the device comprises an array of tactile pressure sensors configured for placement over the artery of the patient. The pressure signals from the individual pressure sensors may be routed through the signal conditioning electronics towards the microprocessor of the control unit of the device. The device may further include battery and power management components to energize the electronics. In other embodiments, wall power may also be used to power up the device.

The control unit may be further operably connected with the indicator of arterial location so as to energize the light positioned next to the individual pressure sensor over the artery.

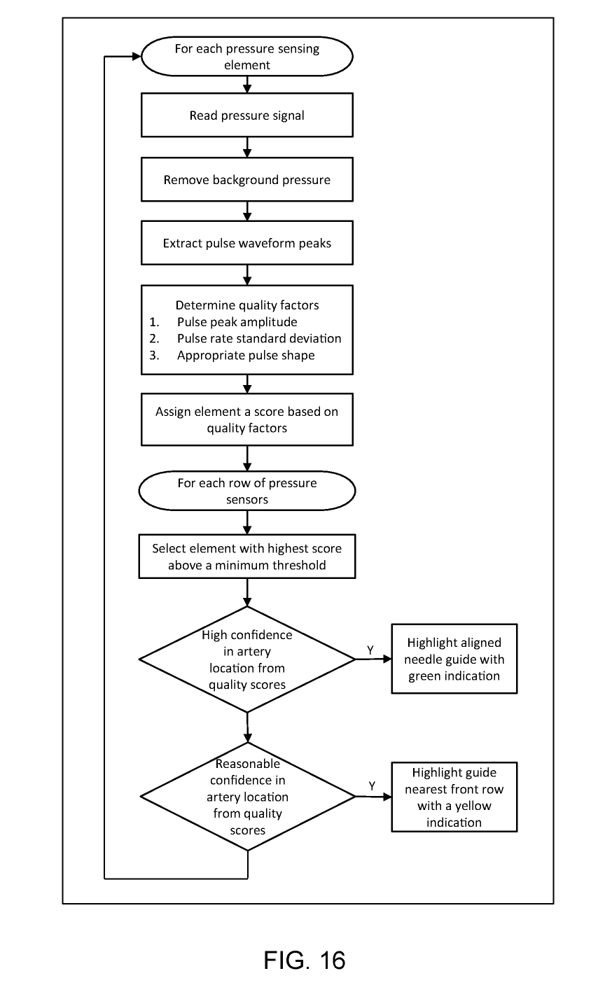

FIG. 16 shows details of signal processing by the control unit of the present invention. The control unit may be configured to read pressure signal from each individual pressure sensor once the device is placed over the presumed location of the artery. Raw signal from each sensor may be first conditioned and processed to remove static background pressure. Pulse waveforms may then be identified and peaks of these pulses may be determined. The processing of the signals then continues to the stage of determining quality factors for each signal from individual pressure sensors. Such quality factors may include at least one or more of pulse peak amplitude, pulse rate standard deviation and appropriate pulse shape. Each row of sensors may be evaluated to identify a sensor with the highest quality factor score. Signals with low levels of quality factors may be discarded or more data may be accumulated to assure acquisition of suitable data to make a reliable determination of the arterial location. A set of predetermined thresholds for each quality factor may be programmed ahead of time for each particular application, such as finding artery in a wrist or finding a femoral artery in the groin area.

Importantly, the device of the present invention includes a set of minimum thresholds for the quality factors. For example, the device may be programmed to expect applied pressure to be greater than about 100 mmHg, reaching about 400 mmHg as a maximum. A minimal peak pressure may be set at about 20 mmHg, with a typical peak pressure expected to reach about 90 mmHg. A physiological range for pulse rate may also be established, for example between about 20 beats per minute and 250 beats per minute. In other words, in order to indicate the location of the artery reliably, it is necessary to not only identify the individual pressure sensor with the highest peak of amplitudes but also it is necessary to pass the minimum threshold in quality factors in order to energize the suitable LED. The control unit may be programmed to proceed with energizing a suitable LED only when high confidence is present in arterial location, for example by using green lights.

In embodiments, when the confidence in arterial location identification is not high but still reasonable, the LEDs may be turned yellow to cause additional attention of the clinician and perhaps double check by manual palpation.

In circumstances with weak pulse, thick subcutaneous tissue or otherwise when sufficient pulsations are not detected, the device may be configured to communicate “failure to detect” state—for example by flashing all LEDs at the same time. Repositioning of the device may then be attempted so as to acquire a better set of signals.

In embodiments, the device may be equipped with a selector switch (not shown) making it suitable for various tasks at hand. The selector switch may have a position for radial artery location, another position for femoral artery location, and other positions for other arterial locations of interest throughout the body. Each position of the switch may be associated with a predetermined set of thresholds and minimum levels of quality factors which are specific for this particular location.

Further embodiments may include further adjustments in these predetermined thresholds to account for example for a child patient or obese patient. Selecting of these parameters may be done using additional selector switches.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A device for identifying arterial location, said device comprising:
a housing comprising side walls joined with a bottom portion and shaped to at least partially cover one or two fingers of a human hand so as to shield from an inadvertent needle puncture,
a tactile pressure sensor array positioned on said bottom portion of said housing facing towards said arterial location, said tactile pressure sensor array comprising an array of at least two lines of between 3 and 100 individual capacitive tactile pressure sensors configured to detect a pressure signal corresponding to a tactile pressure imparted thereon,
a needle guide comprising a first edge extending from said bottom portion and positioned in parallel with at least one line of said individual capacitive tactile pressure sensors, said first edge comprising a plurality of spaced apart first notches sized to facilitate positioning of a needle therein during percutaneous needle puncture,
an indicator of arterial location comprising a linear plurality of lights, each light aligned to be located next to a corresponding notch of said needle guide, and
a control unit operably connected with said tactile pressure sensor array and said indicator of arterial location, said control unit is configured to detect said arterial location using pressure signals using said plurality of individual capacitive tactile pressure sensors, said control unit is further configured to select and energize the light closest to said detected arterial location along said needle guide to identify the closest first notch on said needle guide so as to facilitate said percutaneous needle puncture.

2. The device as in claim 1, wherein said individual capacitive tactile pressure sensors are spaced apart from each other by about 0.01 inches to about 0.40 inches inbetween.

3. The device as in claim 2 further comprising a motion sensor, said control unit is configured to detect a state of the device as stationary or moving using said motion sensor, said control unit is further configured to separate true pressure signals when said device is detected as stationary from motion artifacts when said device is in detected as moving.

4. The device as in claim 2, wherein said motion sensor is an accelerometer or a gyroscope.

5. The device as in claim 2, wherein said needle guide further comprising a second edge extending from said bottom portion and positioned in parallel with said first edge, said second edge is located closer to said bottom portion than said first edge, said second edge comprising a plurality of second notches sized and spaced apart the same as said first notches so as to be aligned therewith, whereby defining an angle of percutaneous puncture when a needle is placed in contact with an adjacent pair of said first notch and said second notch.

6. The device as in claim 2, wherein said lights are light-emitting diodes.

7. The device as in claim 2 further comprising a disposable removable cover configured for purposes or sterility and infection control.

8. The device as in claim 1, wherein said housing has an elongated handle configured to be hand-held.

9. The device as in claim 1, wherein said individual pressure sensors are selected from a group consisting of piezo pressure sensors, piezo-resistive pressure sensors, optical pressure sensors, fiber-optic pressure sensors, resistive pressure sensors, electrical impedance pressure sensors, quantum tunneling pressure sensors, and capacitive tactile pressure sensors.

10. A method for identifying an arterial location and facilitating percutaneous puncture to gain arterial access, said method comprising the following steps:
a. providing a device for identifying arterial location, said device comprising:
i. a tactile pressure sensor array comprising at least two lines of between 3 and 100 individual capacitive tactile pressure sensors configured to detect a pressure signal corresponding to a tactile pressure imparted thereon,
ii. a needle guide comprising a first edge positioned in parallel with at least one line of said individual pressure sensors, said first edge comprising a plurality of spaced apart first notches,
iii. an indicator of arterial location comprising a linear plurality of lights, each light aligned to be located next to a corresponding notch of said needle guide, and
iv. a control unit operably connected with said tactile pressure sensor array and said indicator of arterial location,
b. positioning said device with said tactile pressure array located near a presumed arterial location,
c. obtaining pressure signals and extracting pulse waveform peaks from each of said plurality of individual capacitive tactile pressure sensors,
d. identifying at least one individual capacitive tactile pressure sensor with a highest quality score above a predetermined minimum threshold, said quality score is determined from said pulse waveforms of said individual capacitive tactile pressure sensors using at least one of a pulse peak amplitude, pulse rate standard deviation,
e. causing said control unit to select and energize the light closest to said at least one individual pressure sensor identified in step (d) to identify the first notch on said needle guide closest to said arterial location so as to facilitate said percutaneous needle puncture.

11. The method as in claim 10, wherein said device in step (a) further comprising a motion sensor, said step (c) further including detecting a state of the device as stationary or moving using said motion sensor, and separating true pressure signals when said device is detected as stationary from motion artifacts when said device is in detected as moving.

12. The method as in claim 10, wherein said step (c) further comprising signal processing steps of removing background pressure to isolate waveform pulses and extracting said pulse waveform peaks.

13. The method as in claim 12, wherein said step (c) further comprising determination of at least one quality factor for each pulse waveform peak.

14. The method as in claim 13, wherein said quality factor is a pulse peak amplitude, pulse rate standard deviation, or a predetermined pulse shape.

15. The method as in claim 13 further comprising assigning at least some of the pressure signals a quality score based in said quality factors.

16. The method as in claim 15, wherein said preferred location for percutaneous puncture is detected using a pressure signal from an individual pressure sensor having a highest quality score and having said quality score above a predetermined minimum threshold.
